(19) United States
(12) Patent Application Publication
(10) Pub. No.: US 2023/0121863 A1
(43) Pub. Date: Apr. 20, 2023

(54) ELECTROSURGICAL INSTRUMENT

(71) Applicant: FULMER INSTRUMENTS, LLC, Mountain Brook, AL (US)

(72) Inventors: Benjamin B FULMER, Mountain Brook, AL (US); Eugene A. EIGHMY, Mountain Brook, AL (US); Lloyd G.B. COOPER, Mountain Brook, AL (US)

(21) Appl. No.: 17/969,427

(22) Filed: Oct. 19, 2022

Related U.S. Application Data
(60) Provisional application No. 63/257,416, filed on Oct. 19, 2021.

(51) Int. Cl. A61B 18/14

(52) U.S. Cl. CPC A61B 18/1442; A61B 2018/1462; A61B 2018/0091

(57) ABSTRACT

A handheld electrosurgical instrument is disclosed, which comprises an upper lobe and a lower lobe, which join at and extend from a narrow neck region substantially perpendicular to one another. The instrument typically includes a surgical tool extending from a distal portion of the upper lobe. The tool may be modular, and the instrument may accommodate any of a plurality of different types and configurations of surgical tools.

### PRIORITY CLAIM

This application claims priority to and the benefit of Provisional Application No. 63,257,416, filed Oct. 19, 2021.

### TECHNICAL FIELD

The present disclosure relates generally to electrosurgical devices.

### BACKGROUND

Electronic surgical instruments have been used for cutting and coagulating tissue in surgery since the mid-twentieth century. Typical electrical instruments are wired to a bulky controller and also to the patient. The surgeon, while using the instrument with its wire extending through or across the surgical field, often must work also around tubes, cords, and objects from other surgical devices or systems. This can create a sub-optimal surgical environment. Additionally, some electronic surgical instruments are not ergonomically optimized, and ease of use is compromised. For many surgical procedures, a surgeon must precisely manipulate the instrument using the fine motor control neuromuscular systems of the fingers and hand. Yet with some current designs, the surgeon must at the same time exercise considerable muscular engagement to stabilize and maintain the overall device in position.

Accordingly, there remains a need in the art for an improved electrosurgical device operable wirelessly and with an ergonomic design facilitating ease of use in the surgical environment.

### BRIEF DESCRIPTION OF THE DRAWINGS

Further features and advantages can be ascertained from the following detailed description that is provided in connection with the drawings described below:

FIG. 1 is a perspective view of an embodiment of the present invention.

FIG. 2 is top view of the embodiment of FIG. 1, without a modular surgical tool in place.

FIG. 3 is a front view of the embodiment of FIG. 2.

FIG. 4 is a side view of the embodiment of FIG. 2.

FIG. 5 is another perspective view of the embodiment of FIG. 1.

FIG. 6 is an exploded perspective view of the embodiment of FIG. 1.

FIG. 7 is a side view of the embodiment of FIG. 1, held in position for use.

FIG. 8 is a perspective view of alternative embodiments of the present invention.

FIG. 9 is a perspective view of another embodiment of the present invention.

FIG. 10 is a side view of the embodiment of FIG. 1 in an equilibrium position when supported at a balance point.

FIG. 11 is a perspective view of the embodiment of FIG. 1 resting in an equilibrium position across the surface between the thumb and index finger of a person's hand, with fingers open.

FIG. 12 is a perspective view of the embodiment of FIG. 1, held in position for use.

FIG. 13 is a side view of the embodiment of FIG. 1, in an equilibrium position and in a second position rotated clockwise from equilibrium.

### DETAILED DESCRIPTION

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terms “about” and “approximately” shall generally mean an acceptable degree of error or variation for the quantity measured given the nature or precision of the measurements. Typical, exemplary degrees of error or variation are within 20 percent (%), preferably within 10%, more preferably within 5%, and still more preferably within 1% of a given value or range of values. Numerical quantities given in this description are approximate unless stated otherwise, meaning that the term “about” or “approximately” can be inferred when not expressly stated.

The terminology used herein is for the purpose of describing particular embodiments only and is not intended to be limiting. As used herein, the singular forms “a”, “an” and “the” are intended to include the plural (i.e., “at least one”) forms as well, unless the context clearly indicates otherwise.

The terms “first,” “second,” “third,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

Spatially relative terms, such as “above,” “under,” “below,” “lower,” “over,” “upper,” and the like, may be used herein for ease of description to describe one element or feature's relationship to another when the apparatus is right side up as shown in the accompanying drawings.

Terms such as “at least one of A and B” should be understood to mean “only A, only B, or both A and B.” The same construction should be applied to longer lists (e.g., “at least one of A, B, and C”).

The term “may” as used herein refers to features that are optional (i.e., “may or may not,”), and should not be construed to limit what is described.

In the drawings and in the description which follows, the term “proximal” will refer to the end of the surgical device which is closest to the operator, while the term “distal” will refer to the end of the device which is furthest from the operator.

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

Embodiments of the present invention provide an electrosurgical instrument preferably configured as a self-contained, hand-held modular device designed with its weight distributed across substantially orthogonally opposed upper and lower lobes to provide a secure, balanced, and controlled use in surgery.

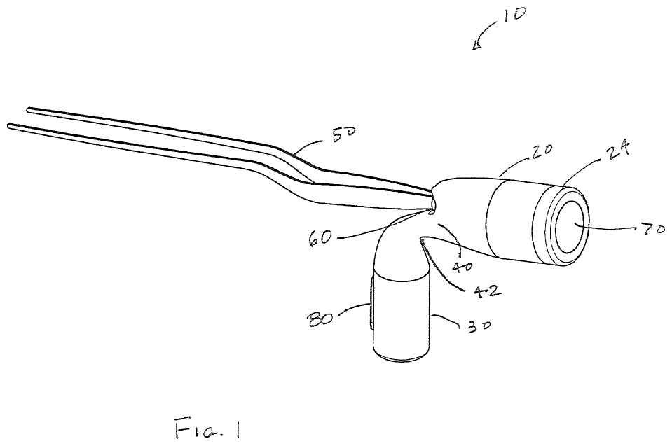

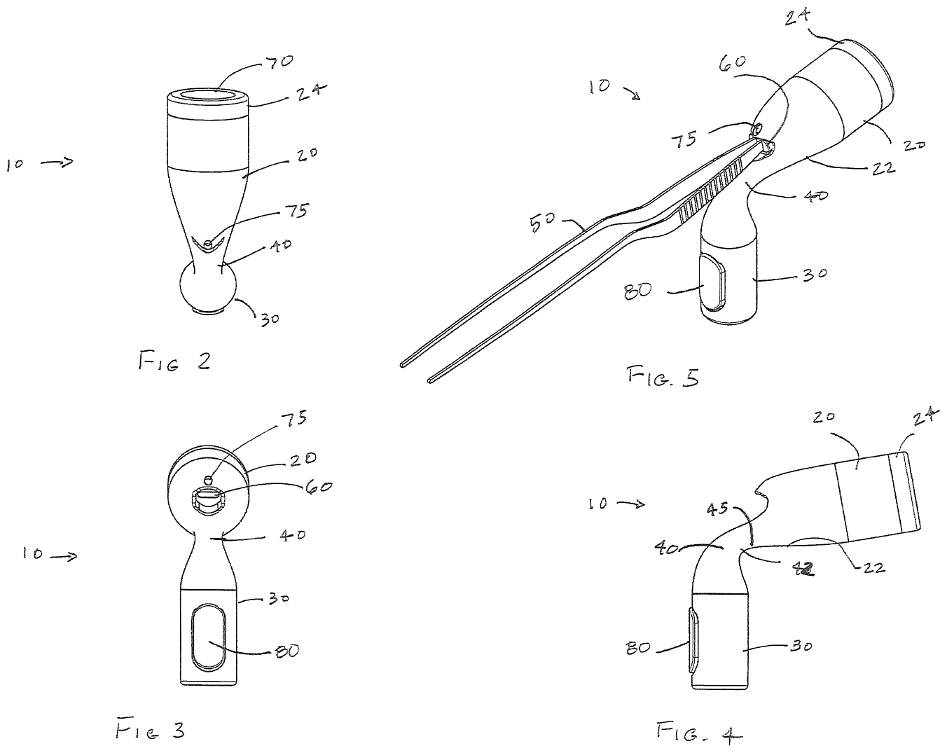

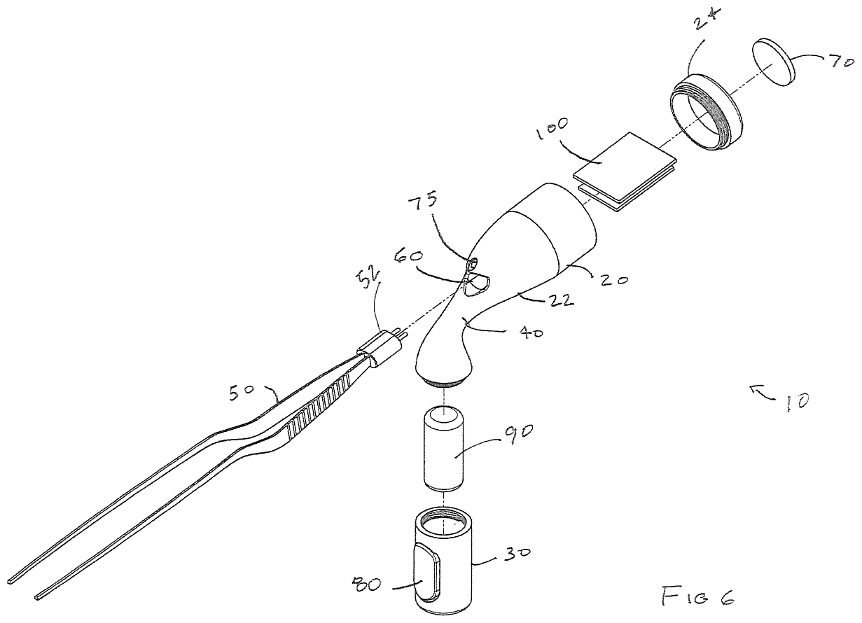

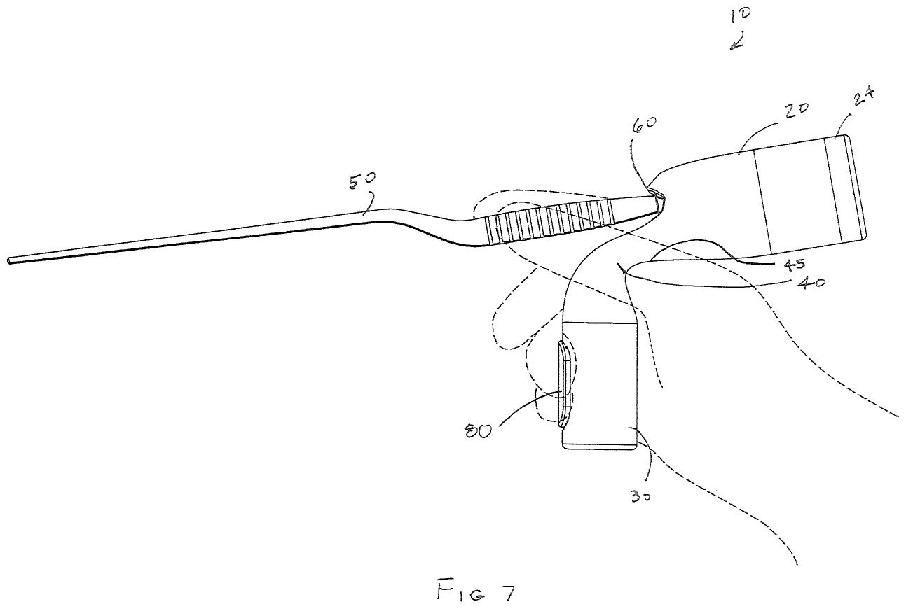

As shown in FIGS. 1-5, an embodiment of the electrosurgical instrument 10 comprises an upper lobe 20 and a lower lobe 30, which join at and extend from a neck region 40 substantially perpendicular to one another. The upper lobe 20 and lower lobe 30 are preferably oblong and more preferably substantially cylindrical in shape, with each lobe oriented about an axis substantially perpendicular to the other. As shown, a lower surface 22 of the upper lobe 20 may be tapered towards a relatively narrow neck 40, which facilitates the ergonomics of the instrument 10 when held. Some or all of the external surface of lower lobe 30 may likewise be contoured for ergonomics to improve feel and security when held.

The configuration of FIGS. 1-5 is a preferred embodiment, and in other embodiments the relative angle between the upper lobe 20 and lower lobe 30 may be more acute or obtuse than shown, with obtuse being preferred, and their shapes altered as described. For example, in one embodiment, the angle between the axes of the upper and lower lobes is about 100 degrees and the upper lobe having a larger diameter than the lower. Hence, as used herein, substantially perpendicular would include the angle between the axes varying from about 80 degrees to about 120 degrees. The surgical instrument 10 also may be provided with adjustability about the neck 40, such that the relative angle between the upper lobe 20 and lower lobe 30 may be customized an individual surgeon's preference. The neck 40 may be provided with an adjustable and lockable joint or may be made with a flexible outer sheath housing a ductile member that can take and maintain a desired position or configuration.

In a preferred embodiment, the electrosurgical instrument 10 includes a modular surgical tool 50 extending from a socket 60 near the distal portion of the upper lobe 20. As shown, the modular tool 50 is a bipolar forceps. However, the module 50 may be any other electrosurgical device desired for a given procedure, such as a scalpel, surgical pencil, ablation probe, catheters or laparoscopic instruments, other monopolar or bipolar devices, drills, fluid vacuums or endoscopic instruments which may be of any size, shape or configuration as desired or required for a given surgical procedure. The modular tool 50 terminates at its proximal end with a connector adapted to be plugged into and removed from the socket 60, which in turn is coupled to the electronics of the electrosurgical instrument 10 described below.

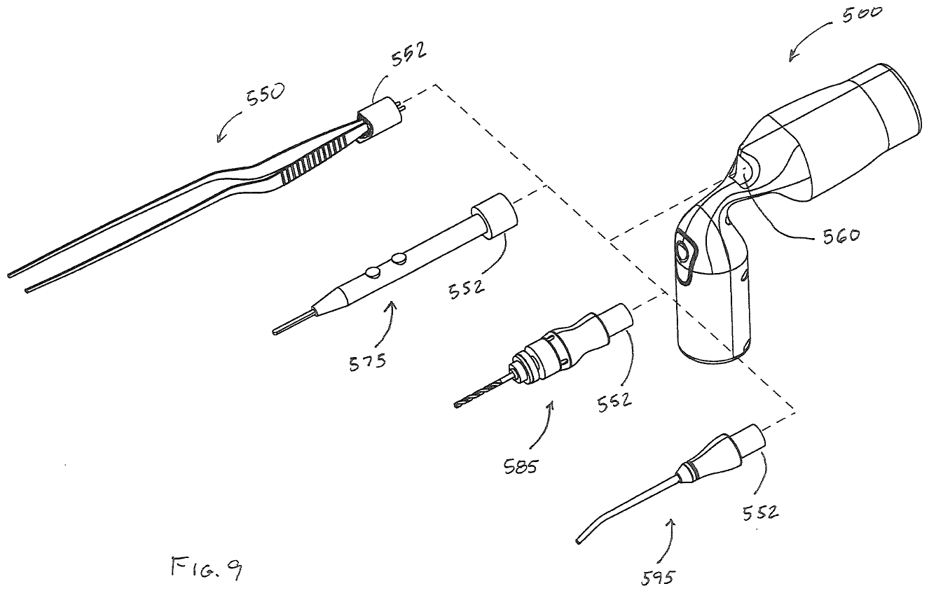

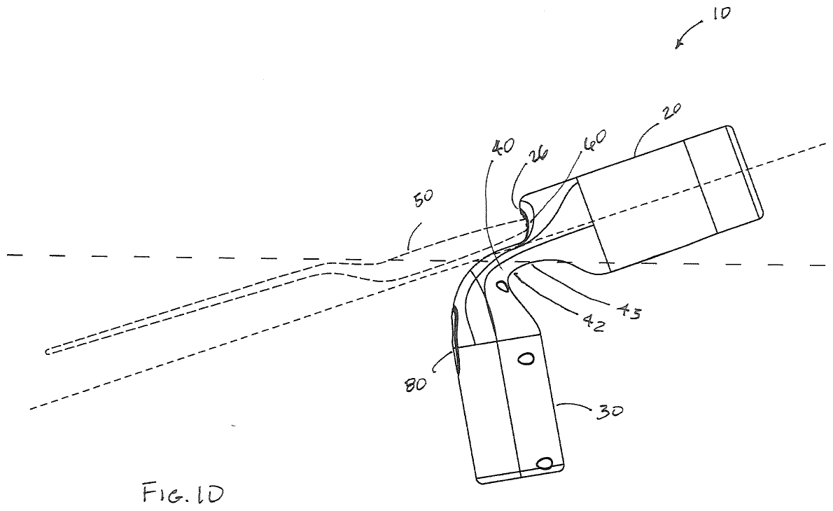

Examples of electrosurgical instruments with a dedicated surgical tool are shown in embodiments 200, 300, and 400 of FIG. 7. Instrument 200 is a bipolar device similar to instrument 100 in a larger form factor. Instrument 300 is a surgical drill, and instrument 400 is a surgical fluid vacuum. A fluid repository 410 may be located at the proximal end of the upper lobe and preferably includes a translucent panel so that fluid level may be visually monitored. As shown, the particular shape and size of the upper and lower lobes in each of the instruments 200, 300, and 400 may be adapted for the particular surgical tool and application while comprising a balanced, two-lobed design as described herein. In yet a further embodiment, as shown in FIG. 9, the instrument 500 including its modular socket 560 is designed to accommodate an enlarged connector 552 that is adapted to support and actuate dedicated functional modules within the housing of various modular surgical devices, such as bipolar forceps 550, modular monopolar cutting tool 575, modular drill 585, or modular vacuum 595. Other modular surgical tools could be provided.

In some embodiments, the proximate end of the upper lobe 20 may be provided with a display 70. The display 70 is directly and naturally in the surgeon's field of vision, but without obstructing the line of sight to the surgical site, when the electrosurgical instrument 10 is in use. The electrosurgical instrument may preferably include a camera 75, or camera and light source (such as an LED), on the distal end of the upper lobe 20. The camera 75, in conjunction with associated circuitry (including in an electronics module 100 described below), is configured to capture and provide imagery, such as real-time surgical imagery, to the display 70.

The lower lobe 30 serves as a grip or handle for the electrosurgical instrument 10. The lower lobe 30 is sized and shaped to fit comfortably within the palm of a hand. The lower lobe 30 is preferably configured as a modular element of the instrument 10, and accordingly lower lobes 30 of varying size and shapes may be provided to suit individual preferences. The lower lobe 30 includes an actuator or trigger 80 to turn the instrument 10 on or off or to change its mode of operation. In the embodiment shown, the actuator 80 is provided as a pressure-sensitive tactile pad, which may be programmable to provide a desired input to the electronics of instrument 10 (described below) in response to a particular gesture (such as pressure in a particular location or duration or sequence). In other embodiment, the lower lobe 30 may include one or more buttons preferably under a polymer cover. In a preferred embodiment, the trigger 80 is a mechanical actuator allowing for continuously variable adjustment with an electrical limit. The lower lobe 30, and preferably within proximate to the actuator 80, may also include one or more sensors to process input from the user, and one or more feedback devices (including haptic) to convey information regarding and allow control of energy delivery through the modular surgical tool 50 into the patient's tissue.

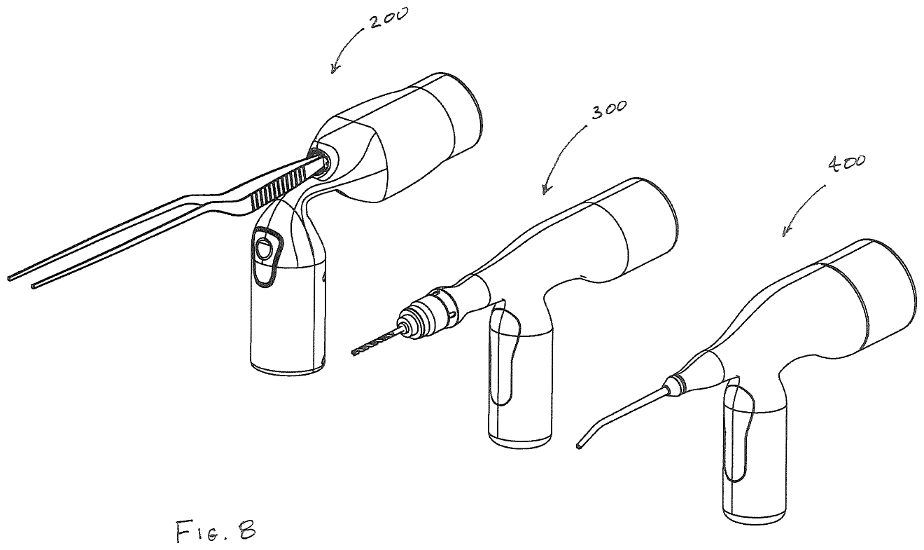

Referring to FIG. 6, the lower lobe 30 typically houses a power source such as a battery 90. The battery 90 may be a lithium polymer battery, or other high-density battery technology now known or hereafter developed and is preferably rechargeable. In some embodiments, the lower lobe 30 is removably attachable to the neck 40, for example by a threaded connection, to allow easy access to the battery 90. An electronics module 100 may be housed within the upper lobe 20. The upper lobe 20 may include a removably attachable end cap 24, which both serves as a mount for the display 70 and also to secure the electronics module 100 within the upper lobe 20. A connector 52, configured to securely and operationally engage in the socket 60, is visible on modular tool 50.

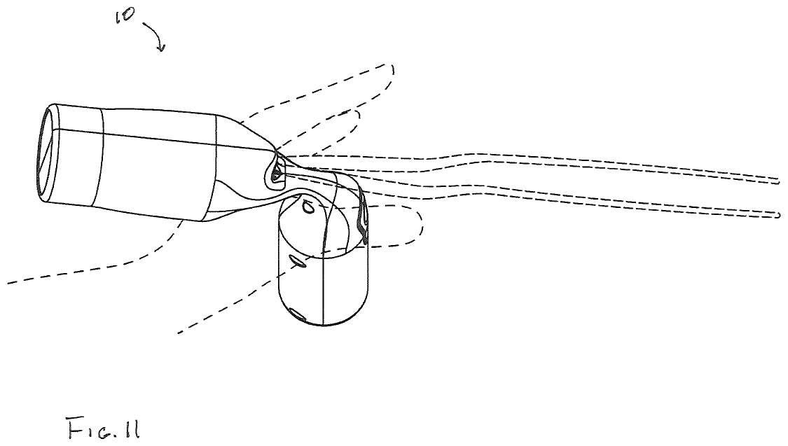

As shown in each of the figures, and in particular in FIGS. 1, 4, and 10, surfaces on each of the upper lobe 20 and lower lobe 30 taper to a narrow neck 40. The ratio of a cross sectional dimension of the lower lobe 30 to that of the neck 40 (such as diameter, when round) may be between 2:1 and 5:1, and more preferably between and including 3:1 to 4:1. The taper and curvature of surfaces at a distal portion of the underside of the upper lobe 20 and a proximate portion of the back of the lower lobe 30 form an arcuate, concave profile 42 of a generally open C shape, which serves as a catch for the instrument 10 to rest in the crook of a surgeon's hand between the thumb and index finger, as described in more detail below. In some embodiments, and as shown in FIG. 10, an angle taken in a section between a surface of the distal underside of the upper lobe and a surface of the proximate back of the lower lobe is acute, while the angle between the longitudinal axes of the upper and lower lobes more generally is obtuse. Preferably, the upper lobe transitions to the neck at a shoulder 26 in which the socket 60 is situated, which in turn places a proximal portion of the modular surgical tool 50 within reach of the surgeon's thumb and index finger for control.

Weight is distributed advantageously across the orthogonally opposed, two-lobed design of the preferred embodiment of FIGS. 1-7, such the instrument 10 will balance at a point between the upper lobe 20 and lower lobe 30 proximate the neck 40. That is, the instrument maintains an equilibrium position if supported only at the balance point. In an embodiment as shown in FIG. 10, in which a balance point 45 is more specifically proximate the junction between the neck 40 and upper lobe 20, the long axis of the upper lobe 20 is elevated between about fifteen and thirty degrees above a horizontal reference in the equilibrium position, and more preferably about twenty degrees above the horizontal reference.

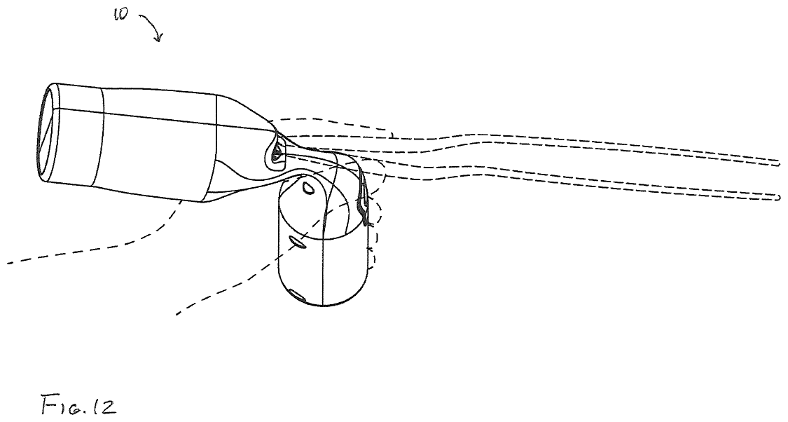

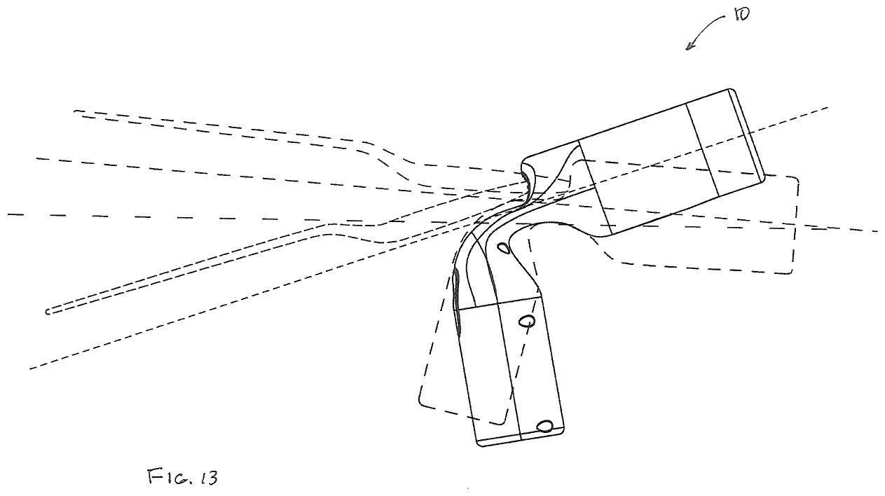

The weight distribution of the instrument 10 and the resulting balance point provide significant ergonomic advantages. As shown in FIG. 11, when balanced with the neck of instrument 10 across the crook of a surgeon's hand between the thumb and forefinger (sometimes referred to as the webspace), with the hand and wrist in a neutral posture relative to the forearm, the equilibrium position places the lower lobe 30 in close proximity or in contact with the palm of the hand and the upper lobe 40 above the back of the hand, clear of the wrist, and extending generally in the direction of the surgeon's forearm. The surgical device 50 extends away from the hand slightly declined relative to the forearm. As a result, the instrument 10 rests across the webspace in stable equilibrium close to the position in which it will be used in surgery, with surgical tool 50 extending towards the surgical site and the display 70 in the surgeon's field of vision. As shown in FIG. 12 and also in FIG. 7, the surgeon's lower fingers may close naturally over the lower lobe 30, which fits within the palm of the hand and is held securely in place with relatively light pressure from the surgeon's lower fingers. Any or all of the second, third, and fourth fingers may engage with the control surface 80, according to the particular tool, controls provided, or surgeon's preference. With the upper and lower lobes 20 and 30 held and positioned as described, the surgeon's thumb and forefinger are free to grasp and control the surgical tool 50 (forceps as shown) with a high degree of dexterity and precision.

The weight distribution and balance of the instrument 10 advantageously allows a self-stabilizing effect, in which the device tends to return to the equilibrium position. That is, if the upper or lower lobe is subject to a perturbance that displaces the device from the equilibrium position, the other lobe provides a restoring force that acts against the perturbance and (up to a limit) returns the instrument to the equilibrium position when the perturbance is removed. This is illustrated in FIG. 13, which shows the instrument 10 in its equilibrium position and in second position (shown in dashed lines) rotated clockwise from equilibrium by a perturbance. The extent of displacement from which the instrument will still return to its equilibrium position depends upon the relative weight distribution between the upper and lower lobes and among the components within them, in a given embodiment. In general, however, when the instrument 10 is in use, either the surgeon's palm, or the back of the hand or wrist, will obstruct movement of the lower lobe 30 or upper lobe 20 of the instrument 10 beyond the range at which it will not tend to return to the equilibrium position. This self-stabilizing effect enhances the security and ease of use in practice. In some embodiments, the instrument may rotate up to 45 degrees clockwise and counterclockwise (referenced as shown in FIG. 13) before the tipping point is reached, and in other embodiments, this range may be reduced to 20 degrees in either direction to provide sufficient self-stabilization in typical use.

In the embodiment shown in FIGS. 1-7, the battery 90 in the lower lobe 30 has greater mass than the electronics module 100 and other components housed in the upper lobe 20. In other embodiments, depending on the surgical tool 50 to be used and its requirements for battery capacity, mechanical components and electronics, these elements could be arranged or configured differently between the upper and lower lobes. In each of these embodiments, the object is to place the center of mass of the electrosurgical instrument within or in close proximity to the neck region of the instrument, which in turn places the center of mass in close proximity to the palm of the surgeon's hand. In general, the upper lobe 20 counterbalances the modular surgical tool 50. The particular location of the center of mass may vary depending on the modular surgical tool 50 in use, yet because of the two-lobed design and other features described herein, the electrosurgical instrument 10 maintains a sense of lightness and balance in use. Similarly, the two-lobed design provides a housing for the necessary functional components of the electrosurgical instrument 10 in a relatively compact space that is roughly congruent with that of the surgeon's hand. This natural size in combination with the location of the center of mass also enhances control of the instrument 10.

In a preferred embodiment, the electronics module 100 includes a microprocessor-controlled signal generator, amplifier, and associated circuitry, in conjunction with the battery 90, to provide a signal having a desired power (voltage and current), frequency, and waveform through the surgical tool 50 and across a patient's tissue at a surgical site to perform a desired surgical process, such as cutting, coagulating, cauterizing, singing, sealing, or fusing, as the case may be, and in accordance with the type of surgical tool 50 in use. The electrosurgical instrument 50 is configured to detect whether a bipolar or monopolar surgical tool 50 is connected to the socket 60 and adapt accordingly. When a bipolar surgical tool 50 is used, current flows out one electrode through the patient's tissue and back via the other electrode. When a monopolar surgical tool 50 is used, current flows out of the monopolar electrode, through the patient's tissue and into a conductive pad in contact with patient's tissue. The pad includes a transmitting antenna which allows an electrostatic return path to a receiving antenna in the electrosurgical instrument 10. Exemplary aspects of circuitry to provide such functionality are described in U.S. Pat. No. 11,146,609, also owned by the applicant hereof.

The display 70 of the electrosurgical instrument 10 is configurable to display device status and patent-specific surgical site information, for example, in conjunction with a software-assisted 3D surgical environment. Device status information may include battery capacity, power level, mode of operation (e.g., cut, coagulate, etc.), time remaining in mode of operation, and indicators of other control settings. A memory, processor, and associated software in the electronics module 100 may be provided with information regarding a patient and the patient's surgery to assist in surgical planning and execution, for example, by a wireless connection to a surgical planning or virtual surgical environment. Such information may include precise spatial and qualitative data derived from high resolution medical imaging technology, such as tomography or magnetic resonance imaging, regarding the surgical site and procedure. This may include the location and identification of tumors, cancerous tissues, critical nerves and blood vessels, and other surgical landmarks. The electrosurgical instrument 10 may be registered with the surgical system to superimpose such information over real-time imagery obtained from the camera 65 during surgery.

The foregoing description illustrates and describes the apparatuses, processes, manufactures, and other teachings of the present disclosure. Additionally, the disclosure shows and describes only certain embodiments of the apparatuses, processes, manufactures, and other teachings disclosed, but, as mentioned above, it is to be understood that the teachings of the present disclosure are capable of use in various other combinations, modifications, and environments and are capable of changes or modifications within the scope of the teachings as expressed herein, commensurate with the skill and/or knowledge of a person having ordinary skill in the relevant art. For example, while this disclosure has embodiments in conjunction with medical devices, tools, and applications, the teachings of the present invention are also applicable to other handheld tools in which the ergonomics of balance, control, and vision are preferred. Applications include handheld drills, rotary tools, soldering irons, and any other handheld instrument amenable to a body having a two-lobed design, as described herein, with a tool extending therefrom. The embodiments described herein are further intended to explain certain best modes known of practicing the processes, manufactures, and other teachings of the present disclosure and to enable others skilled in the art to utilize the teachings of the present disclosure in such, or other, embodiments and with the various modifications required by the particular applications or uses. Accordingly, the apparatuses, processes, manufactures, and other teachings of the present disclosure are not intended to limit the exact embodiments and examples disclosed herein. Any section headings herein are provided only for consistency with the suggestions of 37 C.F.R. § 1.77 or otherwise to provide organizational queues. These headings shall not limit or characterize the invention(s) set forth herein.

We claim:

1. A handheld electrosurgical instrument comprising an upper lobe and a lower lobe, which join at and extend from a narrow neck region substantially perpendicular to one another.
